(19) United States
(12) Patent Application Publication
(10) Pub. No.: US 2022/0370053 A1
(43) Pub. Date: Nov. 24, 2022

(54) INTRAORAL ASSEMBLY ALLOWING UNINHIBITED ENDOSCOPE AND OXYGEN ACCESS

(71) Applicants: Dennis Tollini, Clarence Center, NY (US); Michael Tollini, Clarence Center, NY (US); Russel J. Grosjean, Orchard Park, NY (US); Edward McHugh, Hamburg, NY (US); Albert Diaz-Ordaz, Clarence, NY (US)

(72) Inventors: Dennis Tollini, Clarence Center, NY (US); Michael Tollini, Clarence Center, NY (US); Russel J. Grosjean, Orchard Park, NY (US); Edward McHugh, Hamburg, NY (US); Albert Diaz-Ordaz, Clarence, NY (US)

(21) Appl. No.: 17/323,167

(22) Filed: May 18, 2021

(51) Int. Cl. A61B 13/00

(52) U.S. Cl. CPC A61B 13/00

(57) ABSTRACT

An intraoral assembly for endoscopy, including a bite bar, including a tubular section including a first end and a second end, a first flange arranged at the first end, a second end arranged at the second end, and a first through-hole, and a tongue restraint, including, a proximal end, a distal end, a top surface, a bottom surface, a first side, a second side, and a hole extending from the proximal end to a location proximate the distal end, wherein the bite bar is adjustably and removably connectable to the tongue restraint.

### FIELD

The invention relates generally to intraoral assemblies for endoscopy or oral surgery, and more particularly, to an intraoral assembly that fixates a patient's mouth, restrains a patient's tongue, and allows endoscope and oxygen access therethrough.

### BACKGROUND

An endoscopy bite block or mouth prop is a wedge-shaped implement used in dentistry for dentists working with children and other patients who have difficulty keeping their mouths open wide and steady during a procedure, or during procedures where the patient is sedated, or for other endoscopy procedures. Bite blocks generally have a rubber-like texture and is typically made from thermoplastic vulcanizate (TPV) material. They come in several different sizes, from pediatric to adult, and are typically ridged as to use the teeth to hold them in place. These or similar procedures may also require the use of a tongue restraint that prevents the tongue from falling into the patient's throat during the procedure while under anesthesia. However, while bite blocks and tongue restraints can be useful, they inhibit the oral cavity such that it is difficult to pass not only an endoscope therethrough, but also an oxygen tube and/or carbon dioxide detection tube. Furthermore, current designs do not allow for fixation of bite blocks and tongue restrains together or for easy adjustability therebetween.

Therefore, there is a long-felt need for an intraoral assembly that allows for adjustable connection between a bite bar and a tongue restraint and allows uninhibited access to the oral cavity for endoscopy, oxygen delivery, and carbon dioxide detection.

### SUMMARY

According to aspects illustrated herein, there is provided an intraoral assembly for endoscopy, comprising a bite bar, including a tubular section comprising a first end and a second end, a first flange arranged at the first end, a second end arranged at the second end, and a first through-hole, and a tongue restraint, including, a proximal end, a distal end, a top surface, a bottom surface, a first side, a second side, and a hole extending from the proximal end to a location proximate the distal end, wherein the bite bar is adjustably and removably connectable to the tongue restraint.

In some embodiments, the tongue restraint is curvilinear toward the bottom surface. In some embodiments, the tongue restraint further comprises a first channel arranged in the first side, and a second channel arranged in the second side. In some embodiments, the bite bar further comprises a second through-hole extending radially outward from and connected to the first through-hole, the second through-hole comprising at least one protrusion operatively arranged to engage at least one of the first channel and the second channel. In some embodiments, the at least one protrusion comprises a first protrusion extending in a first direction and operatively arranged to engage the first channel, a second protrusion extending in a second direction, opposite the first direction, and operatively arranged to engage the second channel, wherein a space is arranged between the first protrusion and the second protrusion. In some embodiments, the tongue restraint further comprises a first notch in the bottom surface and extending from the first side and a second notch in the bottom surface and extending from the second side, and the first protrusion and the second protrusion are operatively arranged to engage the first notch and the second notch, respectively, to align the first protrusion and the second protrusion with the first channel and the second channel, respectively. In some embodiments, the location is an opening in the bottom surface. In some embodiments, the opening is connected to the distal end. In some embodiments, the location is at least one aperture extending from the first side to the second side. In some embodiments, the bite bar further comprises a protruding section extending radially outward from the tubular section, the protruding section comprising at least one through-bore. In some embodiments, the first through-hole comprises a first center axis, and the at least one through-bore comprises a second center axis, the second center axis being arranged at an angle relative to the first center axis, the angle being greater than zero. In some embodiments, the intraoral assembly further comprises a strap operatively arranged to be connected to the bite bar to secure the bite bar to a patient's head. In some embodiments, the intraoral assembly further comprises a port connected to the hole and extending from the proximal end. In some embodiments, the intraoral assembly further comprises a handle extending from the proximal end.

According to aspects illustrated herein, there is provided an adjustable intraoral assembly for endoscopy, comprising a bite bar, including a tubular section comprising a first end and a second end, a first flange arranged at the first end, a second end arranged at the second end, and a first through-hole, and a tongue restraint, including a proximal end, a distal end, a top surface, a bottom surface, a first side comprising a first channel, a second side comprising a second channel, and a hole extending from the proximal end to a location proximate the distal end, wherein the bite bar operatively arranged to be fixedly secured to the tongue restraint such that in a first assembly, the distal end is arranged at a first distance from the bite bar, and in a second assembly, the distal end is arranged at a second distance from the bite bar, the second distance being less than the first distance.

In some embodiments, the bite bar further comprises a second through-hole extending radially outward from the first through-hole, the second through-hole comprising a first protrusion extending in a first direction and operatively arranged to engage the first channel, and a second protrusion extending in a second direction, opposite the first direction, and operatively arranged to engage the second channel, wherein a space is arranged between the first protrusion and the second protrusion. In some embodiments, the tongue restraint further comprises a first notch in the bottom surface and extending from the first side and a second notch in the bottom surface and extending from the second side, and the first protrusion and the second protrusion are operatively arranged to engage the first notch and the second notch, respectively, to align the first protrusion and the second protrusion with the first channel and the second channel, respectively. In some embodiments, the first protrusion and the second protrusion engage the first channel and the second channel to create an interference fit. In some embodiments, the location is an opening in the bottom surface. In some embodiments, the location is at least one aperture extending from the first side to the second side.

According to aspects illustrated herein, there is provided an adjustable intraoral assembly for endoscopy, comprising a bite bar, including a tubular section comprising a first end and a second end and a first through-hole, a first flange arranged at the first end, a second end arranged at the second end, and a protruding section extending radially outward from the tubular section, the protruding section comprising a second through-hole having at least one protrusion, and a tongue restraint, including a proximal end, a distal end, a top surface, a bottom surface, a first side, a second side, at least one channel arranged in at least one of the first side and the second side, and a hole extending from the proximal end to a location proximate the distal end, wherein the bite bar is removably connectable to the tongue restraint via the at least one protrusion engaging the at least one channel.

According to aspects illustrated herein, there is provided a tongue restraint and bite bar assembly comprising a bite bar and a tongue restraint. The bite bar comprises an oval shaped center opening or aperture wherein an endoscope or other device can be manipulated. The bite bar comprises at least one, for example two, protrusions that extend radially inward into the ovular aperture, the protrusions forming slots. The tongue restraint generally comprises a curvilinear plate (i.e., the overall shape is flat and curved). The tongue restraint comprises side channels on both the left and right side when the tongue restraint is positioned, curved down, in the back of the patient's mouth. The tongue restraint comprises a tab or a spoon-shaped handle for positioning the assembly within the patient's mouth. The tongue restraint further comprises a port proximate the tab, the port being connected to a channel that runs along the length of the body of the tongue restraint (i.e., center hole). The port is operatively arranged to be connected to an oxygen source or oxygen machine to supply oxygen to the patient without disrupting the center opening. In some embodiments, the port opens at or proximate the distal end of the tongue restraint. In some embodiments, the port opens within the side channels of the tongue restraint. In some embodiments, the port opens at or proximate the distal end of the tongue restraint and within the side channels of the tongue restraint.

In some embodiments, the tongue restraint further comprises cutouts or notches on the bottom surface. The notches engage protrusions of the bite bar. After such engagement, when the bite bar is displaced in a first axial direction relative to the tongue restraint, the bite bar and the tongue restraint are fixedly secured via friction or interference fit creating a first arrangement. Also after such engagement, when the bite bar is displaced in a second axial direction relative to the tongue restraint, opposite the first axial direction, the bite bar and the tongue restraint are fixedly secured via friction or interference fit creating a second arrangement. In the first arrangement, the intraoral assembly comprises a first length that is longer, for example, for overweight adults. In the second arrangement, the intraoral assembly comprises a second length that is shorter, for example, for children. It should be appreciated that any means suitable for adjustably connecting the bite bar and the tongue restraint may be used, for example, via pins, screws, set screws, dowels, snaps, etc.

These and other objects, features, and advantages of the present disclosure will become readily apparent upon a review of the following detailed description of the disclosure, in view of the drawings and appended claims.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1A is a perspective view of an intraoral assembly, in a first arrangement;

FIG. 1B is a perspective view of the intraoral assembly shown in FIG. 1A, in a second arrangement;

FIG. 2A is a front perspective view of a bite bar;

FIG. 2B is a rear perspective view of the bite bar shown in FIG. 2A;

FIG. 2C is a front elevational view of the bite bar shown in FIG. 2A;

FIG. 2D is a side elevational view of the bite bar shown in FIG. 2A;

FIG. 3 is a front perspective view of a bite bar;

FIG. 4 is a cross-sectional view of the bite bar taken generally along line 4-4 in FIG. 3;

FIG. 5A is a top perspective view of a tongue restraint;

FIG. 5B is a bottom perspective view of the tongue restraint shown in FIG. 5A;

FIG. 5C is a rear elevational view of the tongue restraint shown in FIG. 5A;

FIG. 5D is a front elevational view of the tongue restraint shown in FIG. 5A;

FIG. 5E is a side elevational view of the tongue restraint shown in FIG. 5A; and,

FIG. 6 is a cross-sectional view of the tongue restraint taken generally along line 6-6 in FIG. 5A.

### DETAILED DESCRIPTION

At the outset, it should be appreciated that like drawing numbers on different drawing views identify identical, or functionally similar, structural elements. It is to be understood that the claims are not limited to the disclosed aspects.

Furthermore, it is understood that this disclosure is not limited to the particular methodology, materials and modifications described and as such may, of course, vary. It is also understood that the terminology used herein is for the purpose of describing particular aspects only, and is not intended to limit the scope of the claims.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments. The assembly of the present disclosure could be driven by hydraulics, electronics, pneumatics, and/or springs.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

It should be understood that use of “or” in the present application is with respect to a “non-exclusive” arrangement, unless stated otherwise. For example, when saying that “item x is A or B,” it is understood that this can mean one of the following: (1) item x is only one or the other of A and B; (2) item x is both A and B. Alternately stated, the word “or” is not used to define an “exclusive or” arrangement. For example, an “exclusive or” arrangement for the statement “item x is A or B” would require that x can be only one of A and B. Furthermore, as used herein, “and/or” is intended to mean a grammatical conjunction used to indicate that one or more of the elements or conditions recited may be included or occur. For example, a device comprising a first element, a second element and/or a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element.

Moreover, as used herein, the phrases “comprises at least one of” and “comprising at least one of” in combination with a system or element is intended to mean that the system or element includes one or more of the elements listed after the phrase. For example, a device comprising at least one of: a first element; a second element; and, a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element. A similar interpretation is intended when the phrase “used in at least one of:” is used herein. Furthermore, as used herein, “and/or” is intended to mean a grammatical conjunction used to indicate that one or more of the elements or conditions recited may be included or occur. For example, a device comprising a first element, a second element and/or a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element.

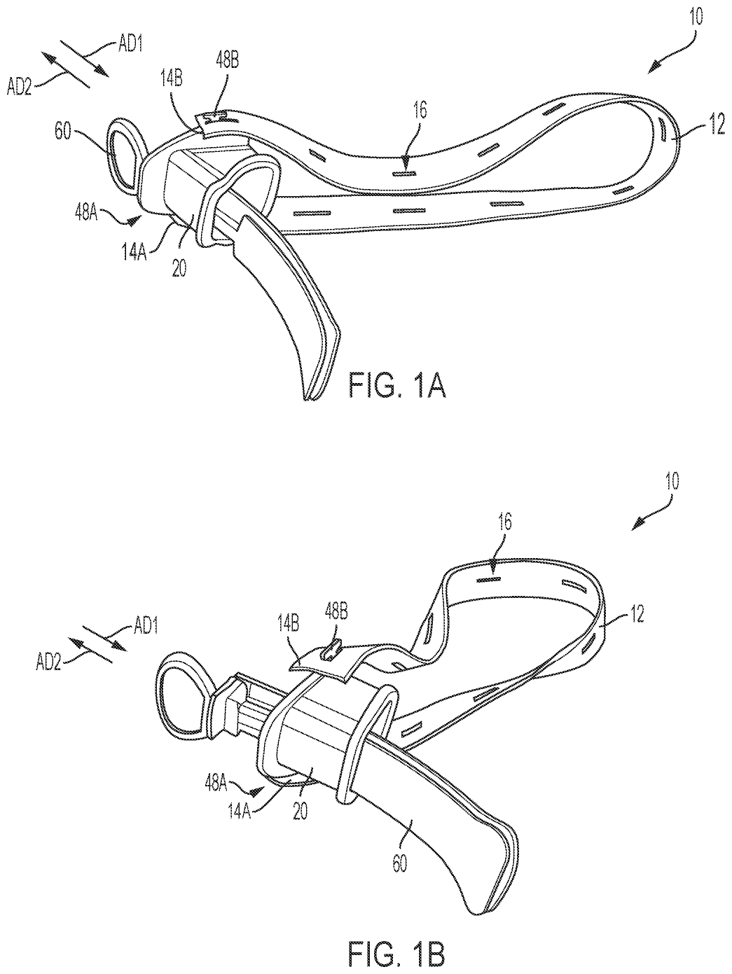

Adverting now to the figures, FIG. 1A is a perspective view of intraoral assembly 10, in a first arrangement. FIG. 1B is a perspective view of intraoral assembly 10, in a second arrangement. Intraoral assembly 10 generally comprises bite bar 20 and tongue restraint 60. In some embodiments, intraoral assembly 10 further comprises strap 12. As shown in FIG. 1A, bite bar 20 is displaced in axial direction AD2 after being engaged with tongue restraint 60. Thus, bite bar 20 is said to be engaged with a proximal end of tongue restraint 60 thereby forming a longer intraoral assembly 10. As shown in FIG. 1B, bite bar 20 is displaced in axial direction AD1 after being engaged with tongue restraint 60. Thus, bite bar 20 is said to be engaged with a central portion of tongue restraint 60 thereby forming a shorter intraoral assembly 10. Such assemblies will become more readily apparent in view of the following description.

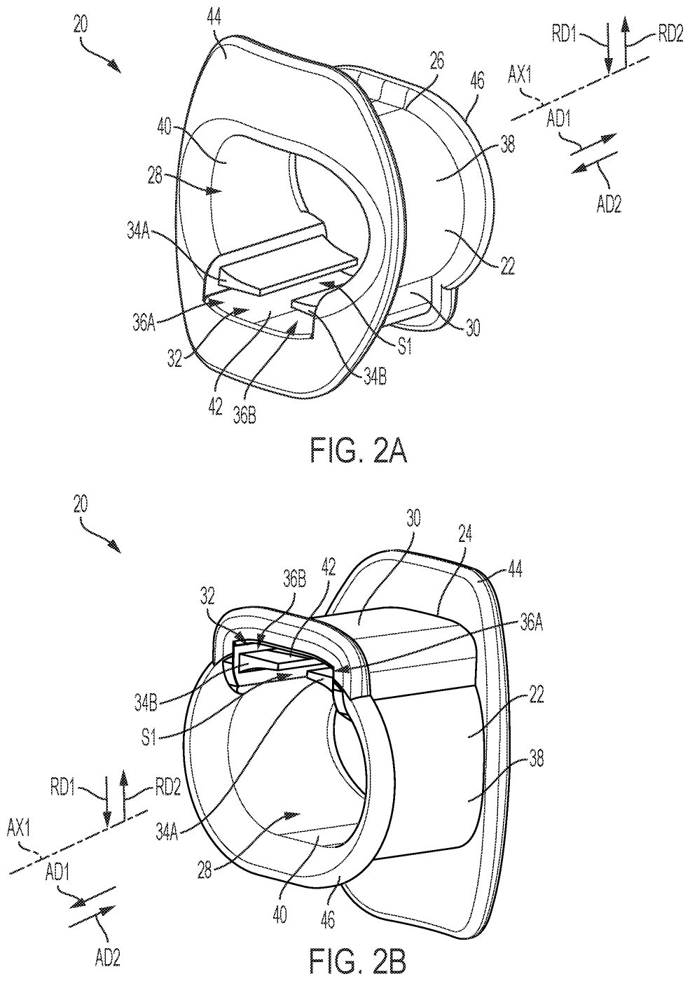

FIG. 2A is a front perspective view of bite bar 20. FIG. 2B is a rear perspective view of bite bar 20. FIG. 2C is a front elevational view of bite bar 20. FIG. 2D is a side elevational view of bite bar 20. Bite bar 20 generally comprises tubular section 22, protruding section 30, radially outward facing surface 38, flange 44, and flange 46. The following description should be read in view of FIGS. 1A-2D.

Tubular section 22 extends from end 24 to end 26 and comprises hole 28 forming radially inward facing surface 40. Hole 28 is a through-hole (i.e., extends completely through bite bar 20) generally comprising an oval or ellipse shaped geometry. The geometry of hole 28, as best shown in FIG. 2C, may comprises a linear top surface and a linear bottom surface, connected via two curvilinear side surfaces (i.e., similar to a super ellipse or a Cassini oval). Hole 28 extends completely through bite bar 20 (i.e., from end 24 to end 26 and through flanges 44 and 46). Axis AX1 runs through the center of hole 28. Hole 28 is operatively arranged to allow a medical professional to insert tools therethrough in order to work on a patient, for example, an endoscope.

Protruding section 30 extends from tubular section 22 generally in a radially outward direction, or in radial direction RD2. Protruding section 30 extends from end 24 to end 26 and comprises hole 32 forming radially inward facing surface 42. The geometry of hole 32, as best shown in FIG. 2C, is substantially rectangular. For example, hole 32 comprises a bottom surface (i.e., radially inward facing surface 42) and two side walls extending therefrom. In some embodiments, radially inward facing surface 42 may be curvilinear. Hole 32 opens into hole 28.

Protruding section 30 further comprises two protrusions 34A-B extending from the two side walls of hole 32. As shown, protrusion 34A extends from a first side wall of hole 32 and is spaced apart from radially inward facing surface 42 forming slot 36A. Protrusion 34B extends from a second side wall of hole 32, opposite the first side wall, and is spaced apart from radially inward facing surface 42 forming slot 36B. Space S1 is formed between protrusion 34A and 34B. Space S1 is operatively arranged to engage notches 74A-B such that protrusions 34A-B can frictionally engage channels 72A-B, as will be described in greater detail below. In some embodiments, protrusions 34A and 34B comprise a curvilinear top surface (i.e., surfaces facing radially inward in radial direction RIM).

Flange 44 is connected to end 24 and extends radially outward therefrom, generally in radial direction RD2. Flange 44 provides a first limit to bite bar 20. Specifically, flange 44 interacts with a patient's lips and/or teeth and/or face to prevent bite bar 20, and intraoral assembly 10, from falling into a patient's mouth or oral cavity. In some embodiments, and as shown, flange 44 is generally a curvilinear plate with hole 28 and hole 32 extending therethrough. The curvilinear shape of flange 44 is such that it resembles the curvature of a patient's face, thus providing a more comfortable and secure fit. In some embodiments, flange 44 is a planar plate with hole 28 and hole 32 extending therethrough.

Flange 46 is connected to end 26 and extends radially outward therefrom, generally in radial direction RD2. Flange 46 provides a second limit to bite bar 20. Specifically, flange 46 interacts with a patient's teeth to prevent bite bar 20, and intraoral assembly 10, from falling out of a patient's mouth or oral cavity. In some embodiments, and as shown, flange 46 is generally a planar plate with hole 28 and hole 32 extending therethrough. In some embodiments, flange 46 is a curvilinear plate with hole 28 and hole 32 extending therethrough.

Tubular section 22 and protruding section 30 form radially outward facing surface 38 of bite bar 20. Radially outward facing surface 38 is operatively arranged to engage a patient's teeth. Specifically, the patient is to bite on radially outward facing surface 38 to maintain the jaw in an open position. Flange 44 and flange 46 work in conjunction to maintain bite bar 20, and thus intraoral assembly 10, aligned with the patient's teeth.

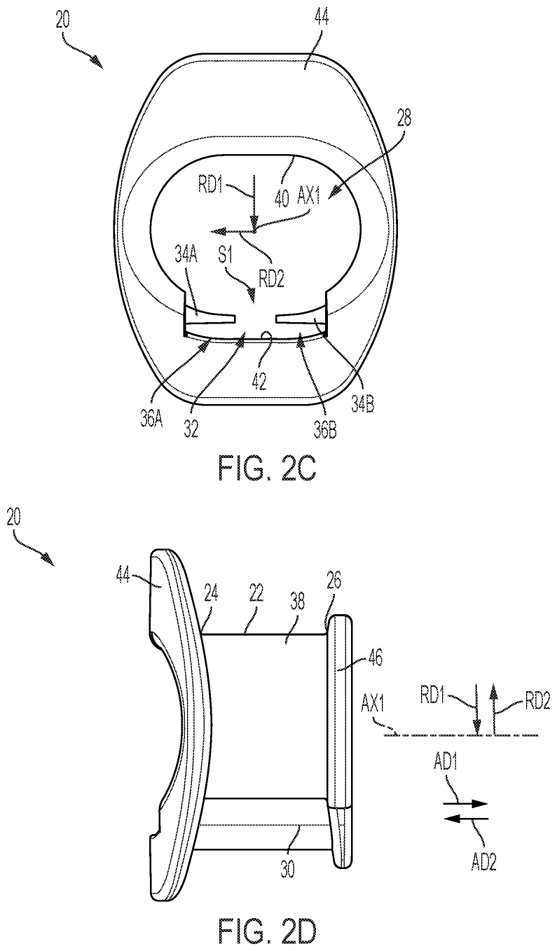

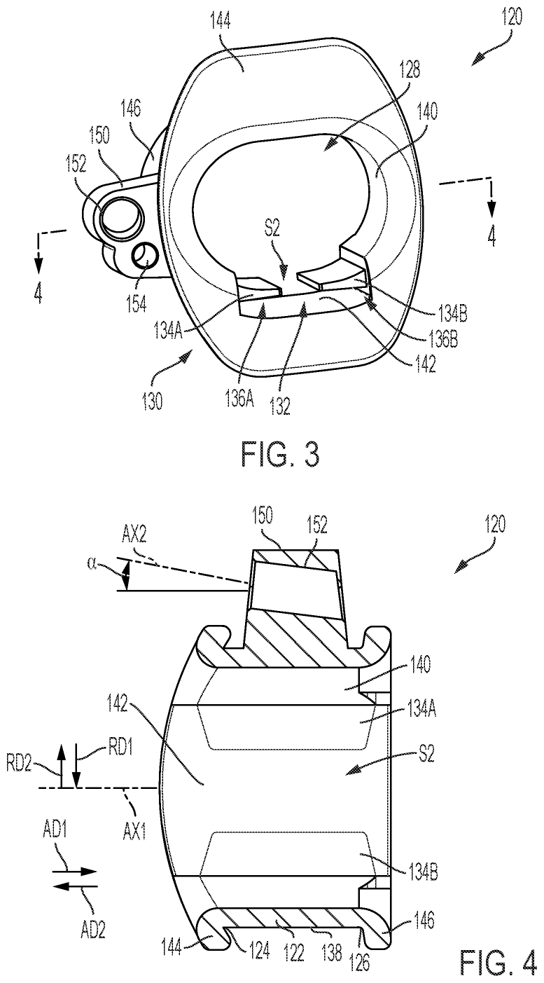

FIG. 3 is a front perspective view of bite bar 120. FIG. 4 is a cross-sectional view of bite bar 120 taken generally along line 4-4 in FIG. 3. Bite bar 120 generally comprises tubular section 122, protruding section 130, radially outward facing surface 138, flange 144, and flange 146. It should be appreciated that, although FIGS. 1A-B depict bite bar 20 connected to tongue restraint 60, bite bar 120 may be used in place of bite bar 20. The following description should be read in view of FIGS. 1A-B and 3-4.

Tubular section 122 extends from end 124 to end 126 and comprises hole 128 forming radially inward facing surface 140. Hole 128 is a through-hole and generally comprises an oval or ellipse shaped geometry. The geometry of hole 128, as best shown in FIG. 3, may comprises a linear top surface and a linear bottom surface, connected via two curvilinear side surfaces (i.e., similar to a super ellipse or a Cassini oval). Hole 128 extends completely through bite bar 120 (i.e., from end 124 to end 126 and through flanges 144 and 146). Axis AX1 runs through the center of hole 128. Hole 128 is operatively arranged to allow a medical professional to insert tools therethrough in order to work on a patient, for example, an endoscope.

Protruding section 130 extends from tubular section 122 generally in a radially outward direction, or in radial direction RD2. Protruding section 130 extends from end 124 to end 126 and comprises hole 132 forming radially inward facing surface 142. The geometry of hole 132, as best shown in FIG. 3, is substantially rectangular. For example, hole 132 comprises a bottom surface (i.e., radially inward facing surface 142) and two side walls extending therefrom. In some embodiments, radially inward facing surface 142 may be curvilinear. Hole 132 opens into hole 128.

Protruding section 130 further comprises two protrusions 134A-B extending from the two side walls of hole 132. As shown, protrusion 134A extends from a first side wall of hole 132 and is spaced apart from radially inward facing surface 142 forming slot 136A. Protrusion 134B extends from a second side wall of hole 132, opposite the first side wall, and is spaced apart from radially inward facing surface 142 forming slot 136B. Space S2 is formed between protrusion 134A and 134B. Space S2 is operatively arranged to engage notches 74A-B such that protrusions 134A-B can frictionally engage channels 72A-B, as will be described in greater detail below. In some embodiments, protrusions 134A and 134B comprise a curvilinear top surface (i.e., surfaces facing radially inward in radial direction RIM).

Protruding section 150 extends from tubular section 122 generally in a radially outward direction, or in radial direction RD2. Protruding section 150 comprises at least one hole, for example, hole 152 and hole 154. Hole 152 comprises center axis AX2 that is nonparallel to axis AX1. As best shown in FIG. 4, axis AX2 is arranged at angle α relative to axis AX1. In some embodiments, angle α is greater than zero. In some embodiments, angle α is 7°. It should be appreciated that hole 152 is arranged at angle α such that a tube inserted therein is directed toward axis A1 and tongue restraint 60, as will be described in greater detail below. In some embodiments, hole 154 comprises a center axis that is parallel to axis AX1. Hole 154 comprises a center axis that is parallel to axis AX1. In some embodiments, hole 154 comprises a center axis that is nonparallel to axis AX1.

In some embodiments, hole 152 comprises a diameter that is greater than the diameter of hole 154. In some embodiments, hole 152 comprises a diameter that is less than or equal to the diameter of hole 154. In some embodiments, hole 152 is operatively arranged to engage a tube connected to an oxygen supply. Such tube is fed through hole 152 in order to deliver oxygen into the patient's mouth, oral cavity, pharynx, esophagus, etc. Thus, the arrangement of hole 152 at angle α relative to axis AX1 allows such a tube to be directed radially inward, generally in radial direction RD1, such that it does not encounter resistance (i.e., get hung up on the roof of the patient's mouth or lining of the esophagus, etc.). In some embodiments, hole 154 is operatively arranged to engage a tube connected to a carbon dioxide detection device. Such tube is fed through hole 154 in order to detect carbon dioxide levels at some point with the patient's body. The other end of the tube, exterior to the patient, is connected to a carbon dioxide detector.

Flange 144 is connected to end 124 and extends radially outward therefrom, generally in radial direction RD2. Flange 144 provides a first limit to bite bar 120. Specifically, flange 144 interacts with a patient's lips and/or teeth and/or face to prevent bite bar 120, and intraoral assembly 10, from falling into a patient's mouth or oral cavity. In some embodiments, and as shown, flange 144 is generally a curvilinear plate with hole 128 and hole 132 extending therethrough. The curvilinear shape of flange 144 is such that it resembles the curvature of a patient's face, thus providing a more comfortable and secure fit. In some embodiments, flange 144 is a planar plate with hole 128 and hole 132 extending therethrough.

Flange 146 is connected to end 126 and extends radially outward therefrom, generally in radial direction RD2. Flange 146 provides a second limit to bite bar 120. Specifically, flange 146 interacts with a patient's teeth to prevent bite bar 120, and intraoral assembly 10, from falling out of a patient's mouth or oral cavity. In some embodiments, and as shown, flange 146 is generally a planar plate with hole 128 and hole 132 extending therethrough. In some embodiments, flange 146 is a curvilinear plate with hole 128 and hole 132 extending therethrough.

Tubular section 122 and protruding section 130 form radially outward facing surface 138 of bite bar 120. Radially outward facing surface 138 is operatively arranged to engage a patient's teeth. Specifically, the patient is to bite on radially outward facing surface 138 to maintain the jaw in an open position. Flange 144 and flange 146 work in conjunction to maintain bite bar 120, and thus intraoral assembly 10, aligned with the patient's teeth.

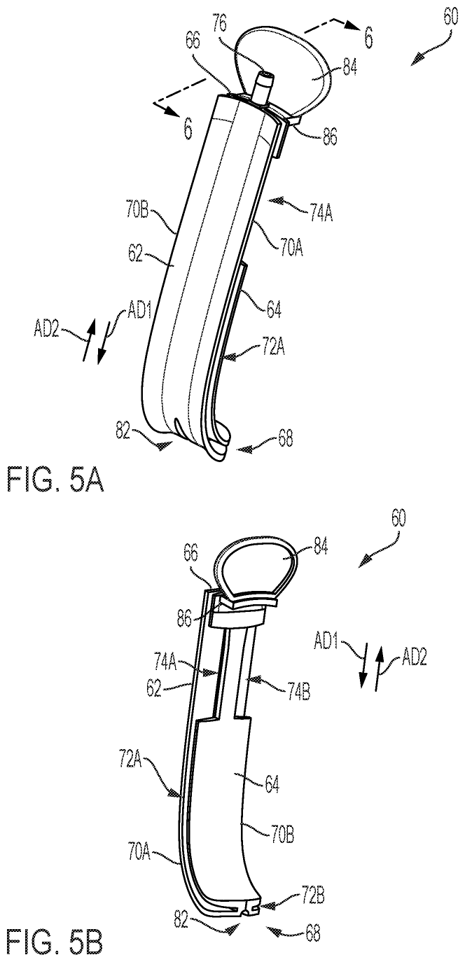

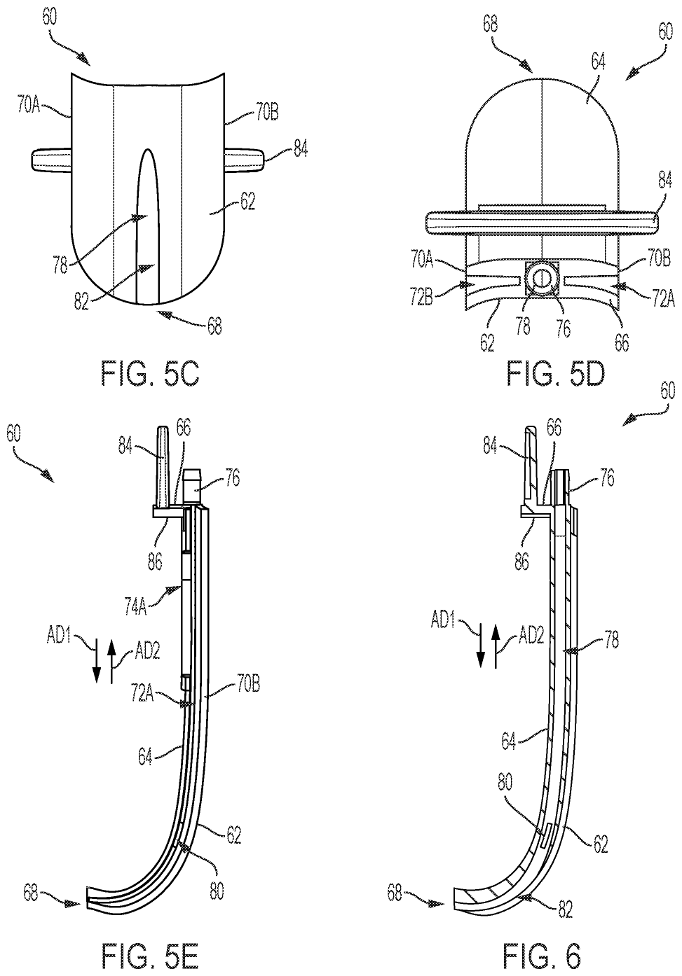

FIG. 5A is a top perspective view of tongue restraint 60. FIG. 5B is a bottom perspective view of tongue restraint 60. FIG. 5C is a rear elevational view of tongue restraint shown 60. FIG. 5D is a front elevational view of tongue restraint 60. FIG. 5E is a side elevational view of tongue restraint 60. FIG. 6 is a cross-sectional view of tongue restraint 60 taken generally along line 6-6 in FIG. 5A. The following description should be read in view of FIGS. 1A-6.

Tongue restraint 60 generally comprises top surface 62, bottom surface 64, proximal end 66, distal end 68, lateral side 70A, and lateral side 70B. Tongue restraint 60 is generally a curvilinear plate. As best shown in FIG. 5E, tongue restraint 60 comprises a generally linear section proximate proximal end 66 and a curvilinear section proximate distal end 68 (i.e., a “J” shape). This specific geometry of tongue restraint 60 is suitable for engaging a patient's tongue, preventing such tongue from displacing into the oral cavity, pharynx, esophagus, etc., and allowing a medical professional to access the workplace without interference therefrom. In some embodiments, distal end 68 may comprise a flared portion or increased width (see FIGS. 1A-B). In some embodiments, tongue restraint 60 comprises a constant width (see FIGS. 5A-D).

Top surface 62 extends from proximal end 66 to distal end 68 and is operatively arranged to face the top of a patient's mouth or oral cavity. Top surface 62 is curvilinear in an axial direction, namely in axial direction AD1, as described above. In some embodiments, and as shown, top surface 62 is curvilinear in a lateral direction (i.e., from side 70A to side 70B). In some embodiments, top surface 62 is planar in a lateral direction (i.e., from side 70A to side 70B).

Bottom surface 64 extends from proximal end 66 to distal end 68 and is operatively arranged to face the bottom of a patient's mouth or oral cavity, namely, to interact with the patient's tongue. Bottom surface 64 is curvilinear in an axial direction, namely in axial direction AD1, as described above. In some embodiments, bottom surface 64 is curvilinear in a lateral direction (i.e., from side 70A to side 70B). In some embodiments, bottom surface 64 is planar in a lateral direction (i.e., from side 70A to side 70B).

Tongue restraint 60 further comprises channels 72A-B which extend inward from sides 70A-B, respectively. Channels 72A-B are operatively arranged to engage protrusions 34A-B, 134A-B, respectively, to fixedly secure bite bar 20, 120 to tongue restraint 60. As best shown in FIG. 5D, each of channels 72A-B comprises a planar bottom and two side walls extending therefrom to sides 70A-B. In some embodiments, the first side wall is planar and perpendicular to the bottom wall and the second side wall is curvilinear. In some embodiments, the first side wall and the second side wall are planar and perpendicular to the bottom wall. In some embodiments, the first side wall and the second side wall are curvilinear. In some embodiments, the first side wall and the second side wall are planar.

Tongue restraint 60 further comprises notches 74A-B which extend inward from sides 70A-B. Notches 74A-B allow protrusions 34A-B, 134A-B to enter channels 72A-B. For example, during assembly of bite bar 20 to tongue restraint 60, space S1 is positioned between notches 74A-B, specifically with protrusion 34A engaging notch 74A and protrusion 34B engaging notch 74, and flange directed toward proximal end 66. Bite bar 20 is then axially displaced in axial direction AD1 or axial direction AD2 with respect to tongue restraint 60. Protrusions 34A-B engage with channels 72A-B on either the side of notches 74A-B toward proximal end 66 or the side of notches 74A-B toward distal end 68. The geometry of protrusions 34A-B and channels 72A-B are such that interaction therebetween forms an interference fit (i.e., the width of channel 72A-B are less than the width of protrusions 34A-B). It should be appreciated that the same assembly procedure can be applied to bite bar 120.

Tongue restraint 60 further comprises hole 78. Hole 78 extends generally through tongue restraint 60 in an axial direction. Hole 78 extends from proximal end 66 in axial direction AD1 toward distal end 68 and is operatively arranged to deliver oxygen into a patient's mouth, oral cavity, pharynx, esophagus, etc. Hole 78 is substantially arranged between and spaced apart from top surface 62 and bottom surface 64, and between and spaced apart from side 70A and side 70B. Hole 78 begins and is open at proximal end 66. In some embodiments, hole 78 is accessible at proximal end 66 via port 76. A tube extending from an oxygen supply may be connected too port 76 to deliver oxygen thereto. In some embodiments, port 76 is integrally formed with tongue restraint 60. In some embodiments, port 76 is a separate element connected to tongue restraint (via, for example, interference fit, adhesives, etc.). Hole 78 ends at a location proximate distal end 68. In some embodiments, hole 78 ends at opening 82, which is an opening that extends from top surface 62 to hole 78. Opening 82 may be arranged at distal end 68 or at a location spaced apart from distal end 68. In some embodiments, opening 82 comprises a “V” shape that extends along top surface 62 from distal end 68. In some embodiments, hole 78 ends at one or more apertures 80. Aperture or apertures 80 are lateral holes that extend through hole 78 from side 70A to side 70B. Thus, hole 78 opens into channels 72A-B. In some embodiments, hole 78 ends at both opening 82 and one or more lateral apertures 80. Use of apertures 80 and/or opening 82 may produce a Venturi effect or Bernoulli's principle. Specifically, the static pressure of the oxygen flowing through hole 78 will decrease after exiting hole 78 through apertures 80 and/or opening 82. The arrangement of hole 78 completely within tongue restraint 60 allows oxygen to be delivered within the patient without interfering with the use of the endoscope or other tools used by the medical professional.

Tongue restraint 60 further comprises axial facing surface 86 proximate the proximal end 66. Surface 86 is directed generally in axial direction AD1 and is operatively arranged to engage and/or abut against flange 44, as shown in FIG. 1A in the first assembly. In some embodiments, surface 86 is curvilinear to match the curvature of flange 44. In some embodiments, surface 86 is planar. Surface 86 acts as a stop or limit for bite bar 20, 120 (i.e., surface 86 limits displacement of bite bar 20, 120 in axial direction AD2 with respect to tongue restraint 60).

In some embodiments, tongue restraint 60 further comprises tab 84 connected to proximal end 66. Tab 84 extends from proximal end 66 in axial direction AD2 and is operatively arranged to act as a handle or holder for tongue restraint 60 and intraoral assembly. A medical professional may connect bite bar 20 to tongue restraint 60, either in the longer first assembly (FIG. 1A) or the shorter second assembly (FIG. 1B), and use tab 84 as a handle to properly position intraoral assembly within the patient's mouth and/or oral cavity.

To use intraoral assembly 10, a bite bar is selected, for example bite bar 20 or 120. It should be appreciated that bite bars 20 and 120 may come in various sizes. For example, a larger bite bar 20, 120 may be selected for a patient having a larger jaw size, whereas a smaller bite bar 20, 120 may be selected for a child patient. As previously described, bite bar 20, 120 is engaged with tongue restraint 60. Specifically, protrusions 34A-B, 134A-B are engaged with notches 74A-B, respectively, with flange 44 directed toward surface 86. Then, based on the desired size of intraoral assembly 10, bite bar 20, 120 is displaced either in axial direction AD1 or axial direction AD2. For a longer intraoral assembly (e.g., for a larger patient), bite bar 20, 120 is displaced in axial direction AD2 (see FIG. 1A). In some embodiments, bite bar 20, 120 is displaced in axial direction AD2 until flange 44 abuts against surface 86. For a shorter intraoral assembly (e.g., for a smaller patient), bite bar 20, 120 is displaced in axial direction AD1. The engagement of protrusions 34A-B, 134A-B with channels 72A-B result in a friction or interference fit, thus securing bite bar 20, 120 to tongue restraint 60. Intraoral assembly 10 is then inserted into the patient. Specifically, distal end 68 is inserted into the patient's mouth with bottom surface 64 directed toward the patient's tongue until bite bar 20, 120 is aligned with the patient's teeth. The patient's teeth are positioned on or engaged with radially outward facing surface 38, 138 and between flanges 44, 144 and 46, 146, with flange 46, 146 arranged internal and flange 44, 144 arranged external to the patient. A tube is then connected to port 76 to supply oxygen to hole 78 and thus to the patient's mouth, oral cavity, pharynx, esophagus, etc. It should be appreciated that the tube may be connected to port 76 after positioning intraoral assembly 10 within the patient or before insertion of intraoral assembly 10 into the patient. Such tube is arranged to be connected to an oxygen supply or source.

In some embodiments, a tube is then inserted into the patient through hole 152 and/or hole 154. The tubes can be engaged with holes 152 and 154 after positioning intraoral assembly 10 within the patient or before insertion of intraoral assembly 10 into the patient. Such tubes may be connected to an oxygen supply and/or a carbon dioxide detection device.

In some embodiments, intraoral assembly 10 further comprises strap 12. As shown in FIGS. 1A-B, strap 12 comprises end 14A, end 14B and a plurality of apertures 16 spaced apart therealong. In some embodiment, bite bar 20, 120 further comprise radially extending connectors or protrusions 48A-B. Connectors 48A-B are operatively arranged to engage apertures 16 to connect strap 12 to bite bar 20 and thus intraoral assembly 10. After apertures 16 are connected to connectors 48A-B, strap 12 is arranged around the patient's head to secure intraoral assembly 10 in position. Apertures 16 are spaced apart along strap 12 such that the length of strap 12 is adjustable based on the size of a patient's head. In some embodiments, connectors 48A-B are “T” shaped. in some embodiments, strap 12 comprises one of male snaps and female straps and connectors comprises the other of male snaps and female snaps. It should be appreciated that intraoral assembly 10 may comprise any means suitable for adjustably connecting strap 12 to bite bar 20. It should be appreciated that in some embodiments, bit bar 20 and tongue restraint 60 are integrally formed.

It will be appreciated that various aspects of the disclosure above and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

### LIST OF REFERENCE NUMERALS

10 Intraoral assembly
12 Strap
14A End
14B End
16 Apertures
20 Bite bar
22 Tubular section
24 End
26 End
28 Hole
30 Protruding section
32 Hole
34A Protrusion
34B Protrusion
36A Slot
36B Slot
38 Radially outward facing surface
40 Radially inward facing surface
42 Radially inward facing surface
44 Flange
46 Flange
48A Connector or protrusion
48B Connector or protrusion
60 Tongue restraint
62 Top surface
66 Bottom surface
68 Proximal end
68 Distal end
70A Side
70B Side
72A Channel
72B Channel
74A Notch
74B Notch
76 Port
78 Hole
80 Aperture or apertures
82 Opening
84 Tab
86 Surface
120 Bite bar
122 Tubular section
124 End
126 End
128 Hole
130 Protruding section
132 Hole
134A Protrusion
134B Protrusion
136A Slot
136B Slot
138 Radially outward facing surface
140 Radially inward facing surface
142 Radially inward facing surface
144 Flange
146 Flange
150 Protruding section
152 Hole
154 Hole
AD1 Axial direction
AD2 Axial direction
AX1 Axis
AX2 Axis
RIM Radial direction
RD2 Radial direction
S1 Space
S2 Space
α Angle

What is claimed is:

1. An intraoral assembly for endoscopy, comprising:
a bite bar, including:
a tubular section comprising a first end and a second end;
a first flange arranged at the first end;
a second end arranged at the second end; and,
a first through-hole; and,
a tongue restraint, including:
a proximal end;
a distal end;
a top surface;
a bottom surface;
a first side;
a second side; and,
a hole extending from the proximal end to a location proximate the distal end;
wherein the bite bar is adjustably and removably connectable to the tongue restraint.

2. The intraoral assembly as recited in claim 1, wherein the tongue restraint is curvilinear toward the bottom surface.

3. The intraoral assembly as recited in claim 1, wherein the tongue restraint further comprises:
a first channel arranged in the first side; and,
a second channel arranged in the second side.

4. The intraoral assembly as recited in claim 3, wherein the bite bar further comprises a second through-hole extending radially outward from and connected to the first through-hole, the second through-hole comprising at least one protrusion operatively arranged to engage at least one of the first channel and the second channel.

5. The intraoral assembly as recited in claim 4, wherein the at least one protrusion comprises:
a first protrusion extending in a first direction and operatively arranged to engage the first channel; and,
a second protrusion extending in a second direction, opposite the first direction, and operatively arranged to engage the second channel;
wherein a space is arranged between the first protrusion and the second protrusion.

6. The intraoral assembly as recited in claim 5, wherein:
the tongue restraint further comprises a first notch in the bottom surface and extending from the first side and a second notch in the bottom surface and extending from the second side; and,
the first protrusion and the second protrusion are operatively arranged to engage the first notch and the second notch, respectively, to align the first protrusion and the second protrusion with the first channel and the second channel, respectively.

7. The intraoral assembly as recited in claim 1, wherein the location is an opening in the bottom surface.

8. The intraoral assembly as recited in claim 7, wherein the opening is connected to the distal end.

9. The intraoral assembly as recited in claim 1, wherein the location is at least one aperture extending from the first side to the second side.

10. The intraoral assembly as recited in claim 1, wherein the bite bar further comprises a protruding section extending radially outward from the tubular section, the protruding section comprising at least one through-bore.

11. The intraoral assembly as recited in claim 10, wherein:
the first through-hole comprises a first center axis; and,
the at least one through-bore comprises a second center axis, the second center axis being arranged at an angle relative to the first center axis, the angle being greater than zero.

12. The intraoral assembly as recited in claim 1, further comprising a strap operatively arranged to be connected to the bite bar to secure the bite bar to a patient's head.

13. The intraoral assembly as recited in claim 1, further comprising a port connected to the hole and extending from the proximal end.

14. The intraoral assembly as recited in claim 1, further comprising a handle extending from the proximal end.

15. An adjustable intraoral assembly for endoscopy, comprising:
a bite bar, including:
a tubular section comprising a first end and a second end;
a first flange arranged at the first end;
a second end arranged at the second end; and,
a first through-hole; and,
a tongue restraint, including:
a proximal end;
a distal end;
a top surface;
a bottom surface;
a first side comprising a first channel;
a second side comprising a second channel; and,
a hole extending from the proximal end to a location proximate the distal end;
wherein the bite bar operatively arranged to be fixedly secured to the tongue restraint such that:
in a first assembly, the distal end is arranged at a first distance from the bite bar; and,
in a second assembly, the distal end is arranged at a second distance from the bite bar, the second distance being less than the first distance.

16. The intraoral assembly as recited in claim 15, wherein the bite bar further comprises a second through-hole extending radially outward from the first through-hole, the second through-hole comprising:
a first protrusion extending in a first direction and operatively arranged to engage the first channel; and,
a second protrusion extending in a second direction, opposite the first direction, and operatively arranged to engage the second channel;
wherein a space is arranged between the first protrusion and the second protrusion.

17. The intraoral assembly as recited in claim 16, wherein:
the tongue restraint further comprises a first notch in the bottom surface and extending from the first side and a second notch in the bottom surface and extending from the second side; and,
the first protrusion and the second protrusion are operatively arranged to engage the first notch and the second notch, respectively, to align the first protrusion and the second protrusion with the first channel and the second channel, respectively.

18. The intraoral assembly as recited in claim 16, wherein the first protrusion and the second protrusion engage the first channel and the second channel to create an interference fit.

19. The intraoral assembly as recited in claim 15, wherein the location is an opening in the bottom surface.

20. An adjustable intraoral assembly for endoscopy, comprising:
a bite bar, including:
a tubular section comprising a first end and a second end and a first through-hole;
a first flange arranged at the first end;
a second end arranged at the second end; and,
a protruding section extending radially outward from the tubular section, the protruding section comprising a second through-hole having at least one protrusion; and,
a tongue restraint, including:
a proximal end;
a distal end;
a top surface;
a bottom surface;
a first side;
a second side;
at least one channel arranged in at least one of the first side and the second side; and,
a hole extending from the proximal end to a location proximate the distal end;
wherein the bite bar is removably connectable to the tongue restraint via the at least one protrusion engaging the at least one channel.
